(19) United States
(12) Patent Application Publication
(10) Pub. No.: US 2003/0171688 A1
(43) Pub. Date: Sep. 11, 2003

(54) Mind controller

(76) Inventors: Jae Sup Yoo, Seoul (KR); Jae Chun Yoo, Seoul (KR)

Correspondence Address: LEE & HONG, 801 SOUTH FIQUEROA STREET, 14TH FLOOR, LOS ANGELES, CA, 90017, US

(21) Appl. No.: 10/199,346

(22) Filed: Jul. 19, 2002

(30) Foreign Application Priority Data
Mar. 6, 2002 (KR) 2002-11846

(51) Int. Cl. A61B 5/04

(52) U.S. Cl. 600/544

(57) ABSTRACT

A mind controller is disclosed. The mind controller can induce a user's brain waves into an alpha wave state or a theta wave state by sensing and analyzing human brain waves and then transmitting a mind control audio message suitable for the analyzed human brain waves to the user, so that the user can improve mental concentration power or memory for himself/herself. The mind controller for activating brain waves generated from the user's brain, includes: an EEG(Electroencephalogram) sensor for sensing frequency band corresponding to alpha waves and theta waves from the brain waves generated from the user's brain; an MCU(Memory Control Unit) for analyzing whether the brain waves sensed by the EEG sensor are alpha waves or theta waves through a built-in program of a brain wave analysis program pack and controlling output of a message, which corresponds to the alpha waves or the theta waves, out of mind control audio messages of an MP3 pack; an audio decoder for demodulating signal converted into data in the MP3 pack by control signal output from the MCU; a D/A converter for receiving signal provided from the audio decoder and converting the signal into analog audio signal; and audio output means for converting and providing the analog audio signal into sound.

### BACKGROUND OF THE INVENTION

1. Field of the Invention

The present invention relates to a mind controller, and more particularly, to a mind controller, which can induce a user's brain waves into an alpha wave state or a theta wave state by sensing and analyzing human brain waves and then transmitting a mind control audio message suitable for the analyzed human brain waves to the user, so that the user can improve mental concentration power or memory for himself/herself.

2. Background of the Related Art

In generally, human brain waves consist of beta waves (14 cycle) of a normal consciousness level, alpha waves (14 cycle˜7 cycle) of an inside consciousness level and theta waves (7 cycle˜4 cycle), and delta waves (less than 4 cycle) of a unconsciousness level. Here, it has been well known that when human brain waves are in an alpha wave state and a theta wave state, people's body and mind can be relaxed and thereby people's memory and judgment can be improved. Also, it has been well known that if such relaxation training in mind and body is continued, people's creative power and intuition are improved.

There are a lot of devices and methods for inducing alpha waves to a user. Out of the prior arts, a method for inducing the alpha waves to the user by giving the user an external stimulus (ultrasonic waves, sound or light) is a method to continuously apply the external stimulus artificially without regard to the state of the user's brain waves.

However, the method has several disadvantages that people cannot confirm that the brain waves are induced into alpha waves actually and the continuous external stimulus is harmful to human body.

### SUMMARY OF THE INVENTION

Accordingly, the present invention is directed to a mind controller that substantially obviates one or more problems due to limitations and disadvantages of the related art.

An object of the present invention is to provide a mind controller, which can induce a user's brain waves into an alpha wave state and a theta wave state by sensing and analyzing human waves and then transmitting a mind control audio message suitable for the analyzed human waves to the user, so that the user can improve mental concentration power or memory for himself/herself.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a mind controller for activating brain waves generated from a user's brain includes: an EEG(Electroencephalogram) sensor for sensing frequency band corresponding to alpha waves and theta waves from the brain waves generated from the user's brain; an MCU(Memory Control Unit) for analyzing whether the brain waves sensed by the EEG sensor are alpha waves or theta waves through a built-in program of a brain wave analysis program pack and controlling output of a message, which corresponds to the alpha waves or the theta waves, out of mind control audio messages of an MP3 pack; an audio decoder for demodulating signal converted into data in the MP3 pack by control signal output from the MCU; a D/A converter for receiving signal provided from the audio decoder and converting the signal into analog audio signal; and audio output means for converting and providing the analog audio signal into sound. The mind controller further includes an LCD(Liquid Crystal Display) connected to the MCU, the LCD displaying the brain state sensed through the EEG sensor and functions selected by a plurality of function buttons.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a configuration view showing an external appearance of a mind controller according to the present invention; and

FIG. 2 is a block diagram of an inside structure of the mind controller according to the present invention.

### DETAILED DESCRIPTION OF THE PREFERRED EMBODIMENT

Reference will now be made in detail to the preferred embodiments of the present invention, examples of which are illustrated in the accompanying drawings.

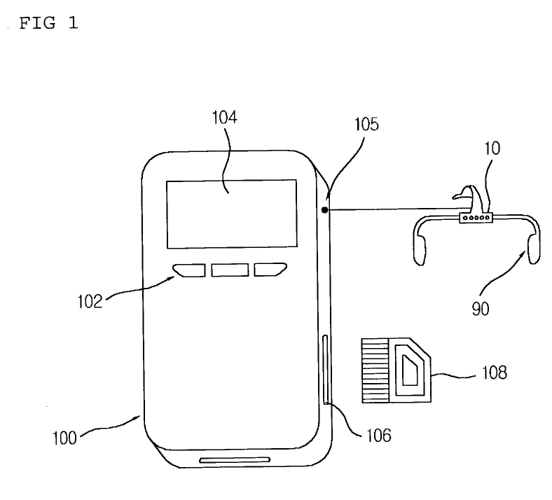

As shown in FIG. 1, a mind controller 100 of the present invention includes a plurality of function buttons 102 and an LCD(Liquid Crystal Display) 104 located at the front surface thereof, and a plurality of connection means located at the side surfaces thereof. A first connection means 105 out of the plurality of connection means is an interface for connecting an earphone or a headphone 90, and a second connection means 106 is an interface for connecting a program pack 108.

The program pack 108 mounted into the mind controller 100 through the second connection means 106 of the mind controller 100 is used for improving mental strength or learning power by inserting the program pack 108 suitable for a user's purpose.

The LCD 104 displays a state of a user's brain waves, such as beta waves, alpha waves, theta waves and delta waves, which are generated from the user's brain, through the mind controller 100.

Furthermore, the mind controller 100 has a dry cell inside or is supplied with electric power from the outside.

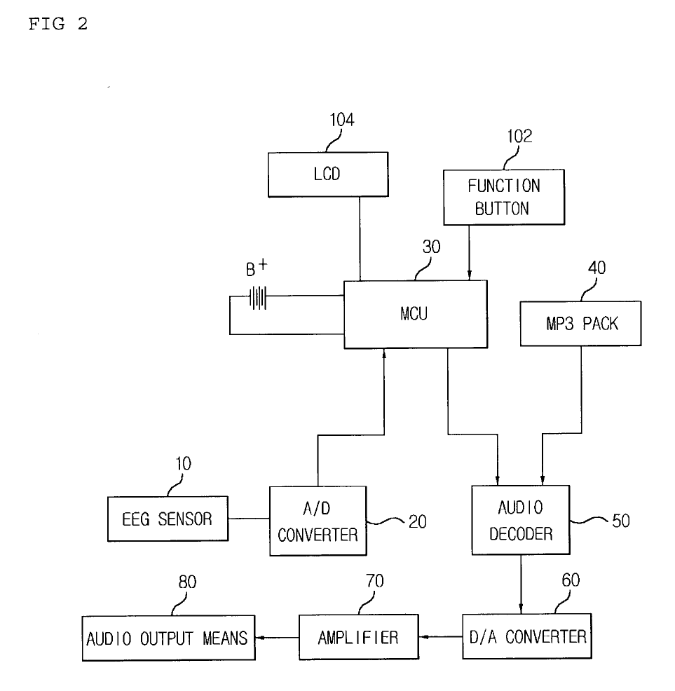

As shown in FIG. 2, the inside of the mind controller includes an EEG(Electroencephalogram) sensor 10, an A/D converter 20, a MCU(Memory Control Unit) 30, an MP3(MPEG Audio Layer-3) pack 40, an audio decoder 50, a D/A converter 60, an amplifier 70, an audio output means 80, and the ear phone or the head phone 90.

The EEG sensor 10 is mounted on the earphone or the headphone 90 to sense weak frequency band generated from the user's brain. The sensed signal is input to the A/D converter 20, the A/D converter 20 amplifies such signal into signal that is capable of being recognized in the MCU 30. The amplified analog signal is converted into digital signal, and the digital signal is input to the MCU 30.

The MCU 30 analyzes whether the user's brain waves are alpha waves or theta waves through the built-in program of the brain wave analysis program pack according to the digital signal level, controls output of a message corresponding to the alpha waves or theta waves out of mind control audio messages of the MP3 pack, displays it on the LCD 104, and operates the audio decoder 50 by the built-in program of the brain wave analysis program pack.

The audio decoder 50 receives and demodulates the mind control audio message data from the MP3 pack 40, converts the demodulated digital signal into analog signal through the D/A converter 60. The converted analog signal is amplified through the amplifier 70, and the audio signal is output to the earphone or the headphone 90 connected through the first connection means 102 of the audio output means 80.

Moreover, it is preferable that the plurality of function buttons 102 disposed on the front surface of the mind controller 100 allow the user to select and play one of the mind control messages provided to the user when the operation of the built-in program of the brain wave analysis program pack is operated.

In the preferred embodiment of the present invention, the mind controller is manufactured in a portable device type for improving and activating mental strength according to the brain wave states, but it will be appreciated that the components used in the present invention are mounted in the existing audio devices, televisions or computers. For example, the device of the present invention can be mounted in the audio device or the computer, and thereby, the same mind control program as the present invention can be carried out.

As described above, the mind controller according to the present invention is manufactured in the portable device type to improve and activate mental strength to reach the alpha wave or theta wave state by operating the mind control program according to the brain wave state. However, the present invention is not restricted to a portable device type, and can be applied to other appliances, such as the audio devices, televisions, computers and so on.

As described above, the mind controller of the present invention induces the user's brain waves into the alpha wave or theta wave state by sensing and analyzing the user's brain waves ad transmitting the mind control audio message suitable for the analyzed brain waves to the user, thereby improving the user's mental strength and learning power, recovering the user from fatigue, removing insomnia and curing patients' diseases.

The forgoing embodiment is merely exemplary and is not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

What is claimed is:

1. A mind controller for activating brain waves generated from a user's brain, comprising:
an EEG(Electroencephalogram) sensor for sensing frequency band corresponding to alpha waves and theta waves from the brain waves generated from the user's brain;
an MCU(Memory Control Unit) for analyzing whether the brain waves sensed by the EEG sensor are alpha waves or theta waves through a built-in program of a brain wave analysis program pack and controlling output of a message, which corresponds to the alpha waves or the theta waves, out of mind control audio messages of an MP3 pack;
an audio decoder for demodulating signal converted into data in the MP3 pack by control signal output from the MCU;
a D/A converter for receiving signal provided from the audio decoder and converting the signal into analog audio signal; and
audio output means for converting and providing the analog audio signal into sound.

2. The mind controller according to claim 1, further comprising an LCD(Liquid Crystal Display) connected to the MCU, the LCD displaying the brain state sensed through the EEG sensor and functions selected by a plurality of function buttons.
